(19) United States
(12) Patent Application Publication
(10) Pub. No.: US 2026/0258400 A1
(43) Pub. Date: Sep. 3, 2026

(54) SYSTEMS AND METHODS FOR STORING INFORMATION IN DNA MOLECULES

(71) Applicant: ROBERT BOSCH GMBH, Stuttgart (DE)

(72) Inventors: Stefan KNOLL, Mountain View, CA (US); Christoph LANG, Sunnyvale, CA (US)

(21) Appl. No.: 19/069,024

(22) Filed: Mar. 3, 2025

(51) Int. Cl. C12N 15/10

(52) U.S. Cl. CPC C12N 15/1065

(57) ABSTRACT

Systems and methods for storing and reading out information are provided. Methods may include synthesizing a DNA molecule having at least one sequence of n nucleotides and at least one sequence of s nucleotides, encoding an element of information in the sequence of n nucleotides, detecting the sequence of n nucleotides, and reading out the information encoded in the sequence of n nucleotides. The sequences of n nucleotides may be labeled with optical or electrical markers capable of being detected by a reader.

### TECHNICAL FIELD

The present disclosure relates to systems and methods for storing information in DNA molecules.

### BACKGROUND

Methods for using DNA to store data have recently been described. The benefit of using DNA as a storage media is the extremely high storage density and the low energy needed to keep the data for a very long time. A disadvantage is that the readout of the data is relatively complicated and time-consuming compared to other state-of-the-art storage methods.

### SUMMARY

In at least an aspect, a method for storing information in a DNA molecule is provided. The method may comprise steps of providing a DNA molecule having at least one sequence of n nucleotides and at least one sequence of s nucleotides; and encoding an element of information in the sequence of n nucleotides to store the element of information in the DNA molecule.

In at least another aspect, a method for reading out information from a DNA molecule is provided. The method may comprise steps of providing a DNA molecule having at least one sequence of n nucleotides and at least one sequence of s nucleotides, wherein the at least one sequence of n nucleotides is labeled with at least one marker to obtain a labeled DNA molecule; detecting the at least one marker; and reading out an element of information encoded in the labeled sequence of n nucleotides.

In yet another aspect, a system for reading out information from a DNA molecule is provided. The method may comprise a device having at least one reservoir; a fluidic chip with at least one channel, the at least one channel in fluid communication with the at least one reservoir; and a channel cover; a voltage source capable of applying an electric field to the at least one channel; at least one reader capable of detecting signals from the device; and a controller. The controller may be programmed to direct the flow of a polymer to the device to promote delivery of the polymer to the surface of the at least one channel. The controller may also be programmed to direct the flow of a sample including a labeled synthesized DNA molecule having at least one sequence of n nucleotides in which information is encoded and at least one spacer sequence of s nucleotides from the at least one reservoir to the at least one channel; generate a voltage difference between an electrode and at least one other electrode to create an electric field in the channel of the device and promote a physical interaction between the labeled synthesized DNA molecule and the polymer to trap the labeled synthesized DNA molecule at the surface of the channel; and interact with the at least one reader to collect, record, store and process a signal received from the at least one reader.

### BRIEF DESCRIPTION OF THE DRAWINGS

FIGS. 1A and 1B illustrate a synthesized DNA molecule according to an embodiment.

FIG. 2 illustrates a labeled synthesized DNA molecule according to an embodiment.

FIGS. 3A and B illustrate a synthesized DNA molecule labeled with multiple markers according to an embodiment.

FIG. 4A illustrates a system for storing and reading out information according to an embodiment.

FIG. 4B illustrates labeled synthesized DNA molecules pinned by a physical interaction with a polymer upon application of an electric field.

FIG. 5A illustrates a system for storing and reading out information according to an embodiment.

FIG. 5B illustrates a system for storing and reading out information according to another embodiment.

FIGS. 6A through 6G illustrate synthesized template DNA molecules for use in incorporating redox labeled nucleotides into a DNA sequence on the template DNA molecule.

FIG. 6H illustrates results of testing showing that methylene blue labeled dUTPs can be incorporated into the template DNA molecules.

### DETAILED DESCRIPTION

As required, detailed embodiments of the present disclosure are disclosed herein; however, it is to be understood that the disclosed embodiments are merely exemplary of the disclosure that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present disclosure.

Except in the examples, or where otherwise expressly indicated, all numerical quantities in this description indicating amounts of material or conditions of reaction and/or use are to be understood as modified by the word “about”. The first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

Unless indicated otherwise, all technical and scientific terms used herein have the same meaning as commonly understood by one of ordinary skill in the art to which the present disclosure belongs.

This disclosure is not limited to the specific embodiments and methods described below, as specific components and/or conditions may, of course, vary. Furthermore, the terminology used herein is used only for describing particular embodiments and is not intended to be limiting in any way.

As used in the specification and the appended claims, the singular form “a,” “an,” and “the” comprise plural referents unless the context clearly indicates otherwise. For example, reference to a component in the singular is intended to comprise a plurality of components.

The terms “or” and “and” can be used interchangeably and can be understood to mean “and/or”.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

The terms “comprising”, “consisting of”, and “consisting essentially of” can be alternatively used. When one of these three terms is used, the presently disclosed and claimed subject matter can include the use of either of the other two terms.

Integer ranges may explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits. In the specific examples set forth herein, concentrations, temperature, and reaction conditions (e.g., pressure, pH, etc.) can be practiced with plus or minus 50 percent of the values indicated rounded to three significant figures. In a refinement, concentrations, temperature, and reaction conditions (e.g., pressure, pH, etc.) can be practiced with plus or minus 30 percent of the values indicated rounded to three significant figures of the value provided in the examples. In another refinement, concentrations, temperature, and reaction conditions (e.g., pH, etc.) can be practiced with plus or minus 10 percent of the values indicated rounded to three significant figures of the value provided in the examples.

In any examples set forth herein, concentrations, temperature, and reaction conditions (e.g., pressure, pH, flow rates, etc.) can be practiced with plus or minus 50 percent of the values indicated rounded to or truncated to two significant figures of the value provided in the examples. In a refinement, concentrations, temperature, and reaction conditions (e.g., pressure, pH, flow rates, etc.) can be practiced with plus or minus 30 percent of the values indicated rounded to or truncated to two significant figures of the value provided in the examples. In another refinement, concentrations, temperature, and reaction conditions (e.g., pressure, pH, flow rates, etc.) can be practiced with plus or minus 10 percent of the values indicated rounded to or truncated to two significant figures of the value provided in the examples.

The terms “polynucleotide”, “nucleotide”, “nucleotide sequence”, “nucleic acid”, “polynucleic acid”, and “oligonucleotide” may be used interchangeably in one or more embodiments this disclosure. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The terms “polynucleotide” and “nucleic acid” may include, as applicable to the embodiment being described, single-stranded (such as sense or antisense) and double-stranded polynucleotides. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term “base” refers to a single nucleotide in one or more embodiments.

The terms “complementarity” or “complement” may refer to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 4, 5, and 6 out of 6 being 66.67%, 83.33%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 40%, 50%, 60%, 62.5%, 70%, 75%, 80%, 85%, 90%, 95%, 97%, 98%, 99%, or 100%, or percentages in between over a region of 4, 5, 6, 7, and 8 nucleotides, or refers to two nucleic acids that hybridize under stringent conditions.

The processes, methods, or algorithms disclosed herein can be deliverable to and/or implemented by a processing device, controller, or computer, which can include any existing programmable electronic control unit or dedicated electronic control unit. Similarly, the processes, methods, or algorithms can be stored as data and instructions executable by a controller or computer in many forms including, but not limited to, information permanently stored on non-writable storage media such as ROM devices and information alterably stored on writeable storage media such as floppy disks, magnetic tapes, CDs, RAM devices, and other magnetic and optical media. The processes, methods, or algorithms can also be implemented in an executable software object. Alternatively, the processes, methods, or algorithms can be embodied in whole or in part using suitable hardware components, such as Application Specific Integrated Circuits (ASICs), Field-Programmable Gate Arrays (FPGAs), state machines, controllers or other hardware components or devices, or a combination of hardware, software and firmware components.

Methods for using DNA to store data have recently been described. The benefit of using DNA as a storage media is the extremely high storage density and the low energy needed to store the data for a very long time. For example, the human genome is composed of about 3 billion base pairs squeezed into a nucleus that is about 10 μm in size. Research suggests that about 215 million gigabytes of data can be stored in 1 gram of DNA which is about 1.5×10¹⁸ base pairs. DNA can therefore store a large amount of information in a very small amount of space. Current digital storage involves using digital data centers with warehouse sized amounts of space and an enormous amount of electricity. In contrast, a gram of DNA takes up less space than a grain of rice. DNA can be stored in a liquid buffer or also in a dry lyophilized state. DNA can be stored in tubes, vials, paper cards, paper discs, and many other small cost-effective easy to store receptacles. Another advantage of using DNA as a storage media is that it provides additional security measures over digital storage methods as the data may only be accessed by sequencing the physical DNA. There is no way to hack into the DNA remotely. A disadvantage of current DNA storage methods is that the readout of the data is relatively complicated and time-consuming compared to other state-of-the-art storage methods. For example, data is currently read out by DNA sequencing methods including sequencing by synthesis which require specialized equipment and costly reagents that must be stored at very specific controlled temperatures. Additionally, these methods require significant expertise to perform. As such there is a need for accessible cost-effective methods for storing information in DNA and for reading it out.

Typically, information stored in DNA is coded in the sequence of the individual nucleotides. For each position in the DNA there are four possible nucleotides: Adenine (A), Cytosine (C), Guanine (G), and Thymine (T). The number of permutations for a DNA segment consisting of n nucleotides is therefore 4ⁿ. In this way, each base pair of DNA can encode one of four states. One of four states would equal two bits of information. If using basic encoding, for example, each nucleotide may be assigned an identifier. For instance, Adenine may be 00, Guanine may be 10, Cytosine may be 01, and Thymine may be 11. This approach allows for the coding of an “element of information” in a single nucleotide. The term “element of information” is meant to describe the elementary component in information coding. In a binary code the element of information would be a bit that can either be 0 or 1. In the case of single-nucleotide encoding, the element of information is the information that can be encoded with one nucleotide. As mentioned above, there are 4 possible elements of information that can be coded with one nucleotide. These elements of information may be combined to express a wider range of data. In the case of binary encoding, 4 bit can encode numbers between 0 and 15. In the case of single-nucleotide encoding, 4 elements of information can encode the numbers from 0 to 255. The information density is huge, because the distance between two bases is on the order of 0.3 nm, which is much smaller than what most modern memories can achieve. Encoding each element of information in a single nucleotide, however, requires sequencing of the DNA molecules at single base pair resolution. Presently, sequencing at this resolution requires costly equipment and reagents and expertise that is generally only present in advanced laboratory settings.

Provided herein are systems and methods for storing information in DNA and for reading it out cost-effectively and without the need for specialized expertise. Methods may include coding each element of information not in the form of single nucleotides but rather in a sequence of n nucleotides. Such methods may result in lower memory density compared to using single nucleotides for information storage but may make the readout possible without the need for DNA sequencing at single-base resolution. In this way, such methods may enable a robust low-cost approach that may be used outside of laboratory type environments and that does not require highly specialized and expensive equipment. As such, there is a need for cost effective methods for storing information methods for storing information in DNA and for reading it out.

In at least an embodiment, a method for storing information in DNA and for reading it out in a cost-effective manner is provided. The method may include synthesizing a DNA molecule including at least one sequence of n nucleotides and at least one sequence of s nucleotides. The at least one sequence of s nucleotides may separate the at least one sequence of n nucleotides. The method may further include encoding an element of information in the sequence of n nucleotides and reading out the information from the synthesized DNA molecule. DNA molecules may be synthesized by non-limiting methods including phosphoramidite synthesis or enzymatic oligonucleotide synthesis for example. Information may be read out from the synthesized DNA molecule by using a reader. In an example, the reader may be a Polymerase Chain Reaction (PCR)-based device rather than a sequencer capable of sequencing DNA at single base pair resolution. In other examples, the reader may be a camera or an electronic nanosensor. Readout technologies may include technologies similar to those used for genome mapping where the reader does not achieve single base resolution but rather is able to identify sequences of base pairs. Such readout technologies will lower the cost of reading out information from the synthesized DNA molecules described herein.

n when referring to the sequence of n nucleotides may be a sequence of varying length. In a non-limiting example, n may be from 6 to 100 nucleotides. The sequence of n nucleotides may be a defined sequence. For example, the sequence of n nucleotides may correspond to the sequence of a particular gene. The sequence of n nucleotides may also be a randomly defined sequence. The sequence of n nucleotides may include an enzyme recognition sequence capable of being recognized by a restriction enzyme or by a nicking enzyme for example. In an example, a sequence of n nucleotides encoding an element of information may include a recognition sequence for a nicking enzyme. The nicking enzyme may generate a nick in one strand of a double stranded linear synthesized DNA molecule. A DNA polymerase such as DNA Polymerase I and fluorescently labeled nucleotides (dNTPs) may be added to a reaction mixture containing the nicked synthesized DNA molecule. The polymerase may then add the labeled nucleotides to the nicked strand and remove the old nucleotides to produce a synthesized DNA molecule with a labeled sequence of n nucleotides. Each synthesized DNA molecule may have at least one sequence of n nucleotides. Where a synthesized DNA molecule has more than one sequence of n nucleotides, each sequence of n nucleotides may have the same number of nucleotides as the others. Alternatively, a sequence of n nucleotides may have a number of nucleotides that is different from another sequence of n nucleotides in the synthesized DNA molecule.

The sequence of n nucleotides may be a randomly defined sequence, a particular gene sequence or gene regulatory element, a sequence representing a combination of genes, or a combination of a gene and/or genetic regulatory elements. Genetic regulatory elements may include but are not limited to a promoter, a terminator, an enhancer sequence, an insulator, or any protein binding sequence. Alternatively, each nucleotide in the sequence of n nucleotides may include the same base. In an example, the sequence of n nucleotides may include a sequence of nucleotides each having a Thymine base. In another example, the sequence of n nucleotides may include a sequence of nucleotides each having a Guanine base. In yet another example, the sequence of n nucleotides may include a sequence of nucleotides each having an Adenine base. In yet another example, the sequence of n nucleotides may include a sequence of nucleotides each having a Cystosine base. Nucleotides having a Uracil base (which pairs with Adenine) may also be used to label a synthesized DNA molecule.

The sequence of s nucleotides may be a spacer sequence. S should be a large enough number so that two sequences of n nucleotides flanking a spacer sequence of s nucleotides may be distinguished from one another during readout. The DNA molecule may include multiple sequences of n nucleotides each separated by a spacer sequence of s nucleotides. Each sequence of n nucleotides may encode one element of information to be stored. Each spacer sequence of s nucleotides does not encode information to be stored. A linear synthesized DNA molecule may begin with either a sequence of n nucleotides or with a spacer sequence of s nucleotides. A linear synthesized DNA molecule may end with either a sequence of n nucleotides or with a spacer sequence of s nucleotides. A synthesized DNA molecule may alternatively be circular. A circular synthesized DNA molecule may include a unique sequence that may be used to linearize the DNA. For example, a circular synthesized DNA molecule may include a recognition sequence for an enzyme including a restriction enzyme that may cut the DNA at the recognition site to linearize the DNA. Multiple sequences of n nucleotides each coding an element of information may each be separated throughout the synthesized DNA molecule by a sequence of s spacer nucleotides. The length of the spacer sequences of s nucleotides may be varied based on the desired resolution of the readout.

The spacer sequence of s nucleotides may be a random sequence. Any of the spacer sequences of s nucleotides may alternatively include one or more specific sequences. The specific sequences present in the spacer sequence of s nucleotides must be different from the information carrying sequences of n nucleotides. Where the element of information encoded in a sequence of n nucleotides is read out via redox molecules, the spacer sequence of s nucleotides may be nucleotides that are different from the nucleotides that are marked with redox molecules. For example, if nucleotides with a Thymine base are marked with redox molecules, the spacer sequence may be nucleotides with a Cytosine, Adenine or Guanine base.

Either end of a linear synthesized DNA molecule may include a primer recognition sequence that may be recognized by a primer used to amplify the entire linear synthesized DNA molecule via a PCR-based amplification method.

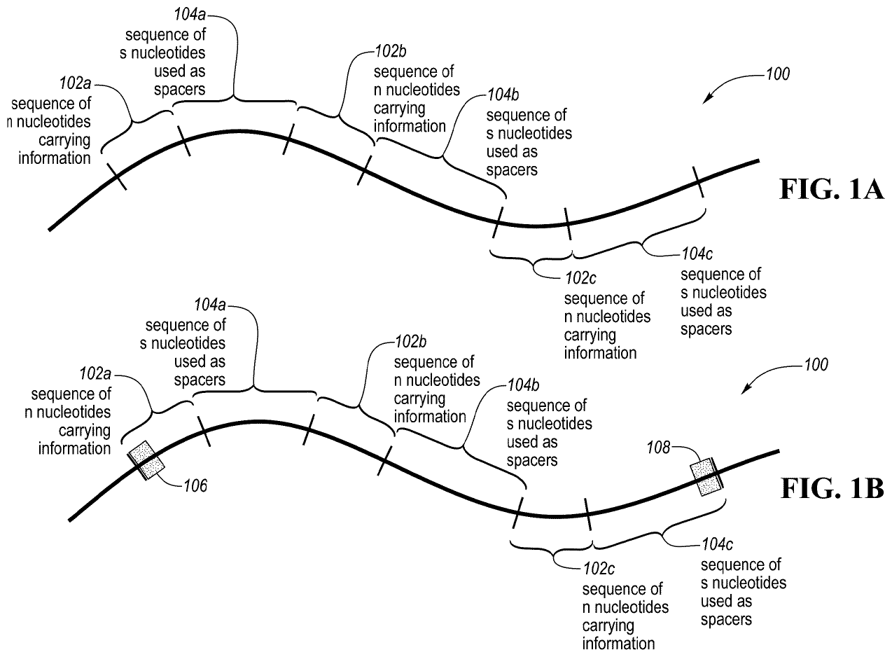

FIG. 1A illustrates a synthesized DNA molecule 100 according to an embodiment. The synthesized DNA molecule may be double stranded. The synthesized DNA molecule 100 includes a sequence of n nucleotides 102a-102c (“102”). The synthesized DNA molecule additionally includes a sequence of s nucleotides 104a-104c (“104”). The sequence of s nucleotides is a spacer sequence. The synthesized DNA molecule includes sequences of n nucleotides 102 separated by spacer sequences of s nucleotides 104.

In one or more embodiments, storing information in a sequence of n nucleotides instead of in a single base pair reduces the readout resolution. In this way, the readout may be accomplished without sequencing the synthesized DNA molecule at single base pair resolution with costly sequencers, expensive and difficult to store reagents, and a high level of expertise.

FIG. 1B illustrates a synthesized DNA molecule 100 according to an embodiment. The synthesized DNA molecule 100 may be double stranded. The synthesized DNA molecule 100 includes a sequence of n nucleotides 102a-102c (“102”). The synthesized DNA molecule 100 additionally includes a sequence of s nucleotides 104a-104c (“104”). The sequence of s nucleotides is a spacer sequence. The synthesized DNA molecule 100 includes sequences of n nucleotides 102 separated by spacer sequences of s nucleotides 104. The sequence of n nucleotides 102a includes an enzyme recognition sequence 106 that may be recognized by a nicking enzyme. This sequence may be used to bring a nicking enzyme to the sequence of n nucleotides 102a to nick the synthesized DNA molecule 100 at the sequence of n nucleotides 102a so that the sequence of n nucleotides 102a may be optically labeled with a fluorescent marker or label for example. The spacer sequence of s nucleotides 104c includes a primer recognition sequence 108. A primer used for amplifying the synthesized DNA molecule 100 or portions of the synthesized DNA molecule 100 or for reading out information stored in the sequences of n nucleotides may recognize the primer recognition sequence and anneal to the sequence to facilitate DNA polymerase activity for example.

In an embodiment, a method for storing information in DNA and for reading it out in a cost-effective manner may include synthesizing a DNA molecule having at least one sequence of n nucleotides and at least one spacer sequence of s nucleotides. The spacer sequence of s nucleotides may include a primer recognition sequence. The primer recognition sequence may be unique to a particular spacer sequence of s nucleotides in the synthesized DNA molecule. The method may also include designing a primer that recognizes the primer recognition sequence in the spacer sequence of s nucleotides and using the primer in a PCR-based amplification reaction to detect the sequence of n nucleotides and read out the information in the sequence of n nucleotides.

According to various embodiments, the sequence of n nucleotides that codes an element of information may be labeled with a marker molecule or several marker molecules of the same kind. The marker molecule may be detectable during readout. The marker molecules may be optical or electrical depending on the chosen readout mechanism. Where a synthesized DNA molecule includes more than one sequence of n nucleotides, each of the sequences of n nucleotides may include the same marker molecule. Alternatively, each of the sequences of n nucleotides may include a different marker molecule. The term label may be used interchangeably with the term marker. The term labeling may be used to refer to the process of incorporating a marker into a sequence of the synthesized DNA molecule.

Optical markers may be attached at a specific sequence of n nucleotides by enzymes. For example, a nicking enzyme may be used to generate a nick in the synthesized DNA molecule and optically labeled nucleotides may be incorporated using a DNA polymerase. This method may be used to label unique or repetitive stretches of DNA. For example, a nicking enzyme may be used to incorporate optically labeled nucleotides into a randomly defined sequence or into a particular gene sequence or gene regulatory element, or into a sequence representing a combination of genes or a combination of a gene and/or genetic regulatory elements. Genetic regulatory elements may include but are not limited to a promoter, a terminator, an enhancer sequence, an insulator, or any protein binding sequence. A single strand of a double-stranded synthesized DNA molecule may be nicked (cut) at a defined location by a nicking endonuclease. Nicking endonucleases recognize a defined DNA sequence and nick the DNA at a specific location within or adjacent to that sequence. Nicking enzymes that may be used for nicking a synthesized DNA molecule at a defined location or locations may include but are not limited to naturally occurring nicking endonucleases such as Nt.BstNB1, Nb.Bts1, or Nb.BsrD1. Nicking endonucleases may additionally include engineered enzymes. Non-limiting examples of engineered enzymes may include Nt.BspQI, Nt.CviPII, Nb.BsmI, Nt.AlwI, Nb.BbvCI, Nb.BssSI, or Nt.BsmAI (produced by New England Biolabs (NEB) of Ipswich, Massachusetts. Nt signifies that the enzyme specifically nicks the top strand of a double stranded DNA molecule. Nb signifies that the enzyme specifically nicks the bottom strand of a double stranded DNA molecule).

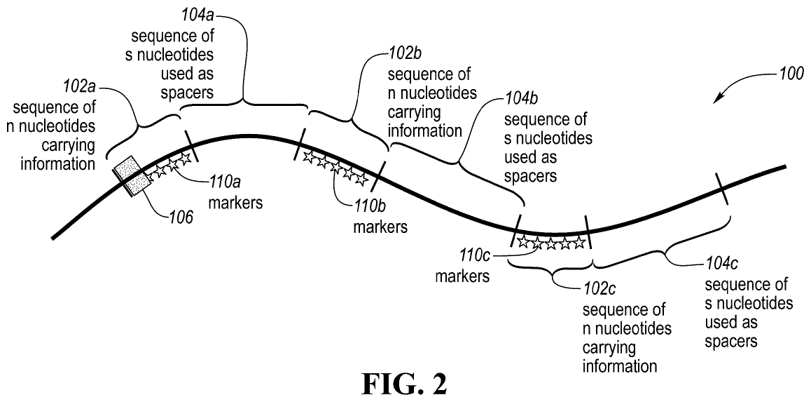

FIG. 2 illustrates a labeled synthesized DNA molecule 100 according to an embodiment. The labeled synthesized DNA molecule may be double stranded. The synthesized DNA molecule 100 includes a sequence of n nucleotides 102a-102c (“102”). The synthesized DNA molecule additionally includes a sequence of s nucleotides 104a-104c (“104”). The sequence of s nucleotides is a spacer sequence. The synthesized DNA molecule includes sequences of n nucleotides 102 separated by spacer sequences of s nucleotides 104. The sequence of n nucleotides 102a includes an enzyme recognition sequence 106 that may be recognized by a nicking enzyme. The nicking enzyme may recognize the enzyme recognition sequence 106 and produce a nick in the synthesized DNA molecule so that optically labeled nucleotides may be incorporated adjacent to the nicked site. The sequences of n nucleotides 102 each include a marker 110a-110c (“110”). The marker may be detectable by a reader. The marker may be detected to facilitate a readout of the information coded into the sequences of n nucleotides.

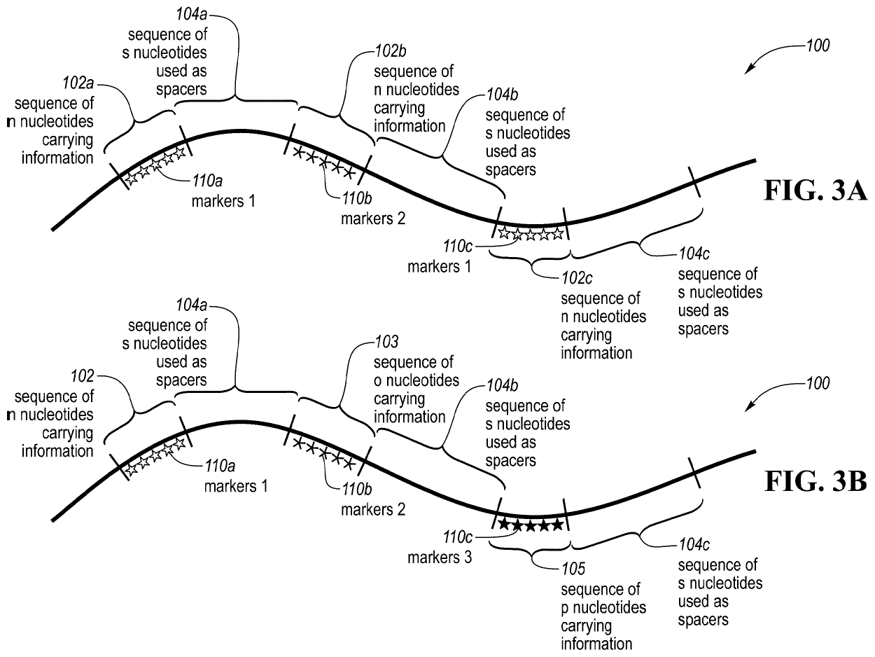

In at least an embodiment, the sequences of n nucleotides in the synthesized DNA molecule may be labeled with different marker molecules depending on the sequence of n nucleotides. For example, with regard to optical markers, several fluorophores with different light transmission wavelengths may be used. FIG. 3A illustrates a synthesized DNA molecule 100 according to an embodiment. The synthesized DNA molecule 100 includes a sequence of n nucleotides 102a-102c (“102”). The synthesized DNA molecule additionally includes a sequence of s nucleotides 104a-104c (“104”). The sequence of s nucleotides is a spacer sequence. The synthesized DNA molecule includes sequences of n nucleotides 102 separated by spacer sequences of s nucleotides 104. The sequences of n nucleotides 102 each include a marker 110a-110c (“110”). In this example, markers 110a and 110c are the same fluorophore. Marker 110b is a different fluorophore with a different light transmission wavelength than that of markers 110a and 110c. Non-limiting examples of optical markers include Alexa fluors, Cy dyes, 7-amino-4-methylcoumarin-3-acetic acid (AMCA acid), 7-Diethylaminocoumarin-3-carboxylic acid (DEAC acid), haptens such as fluorescein, biotin, digoxigenin, and rhodamine, ATTO dyes from ATTO-tec of Siegen, Germany.

FIG. 3B illustrates a synthesized DNA molecule 100 according to an embodiment. The synthesized DNA molecule 100 includes a sequence of n nucleotides 102, a sequence of o nucleotides 103, and a sequence of p nucleotides 105. The sequence of n nucleotides may be of a defined length. The sequence of o nucleotides may be of a defined length that is different from the defined length of the sequence of n nucleotides. The sequence of p nucleotides may be of a defined length that may be different from the defined length of the sequences of n or o nucleotides. The sequences of n, o, and p nucleotides may each be labeled with a unique marker having a light transmission wavelength that may be distinguished from the marker of any of the others. The synthesized DNA molecule additionally includes a sequence of s nucleotides 104a-104c (“104”). The sequence of s nucleotides is a spacer sequence. The synthesized DNA molecule includes sequences of n nucleotides 102 separated by spacer sequences of s nucleotides 104. The sequences of n nucleotides 102 each include a marker 110a-110c (“110”). In this example, markers 110a, 110b, and 110c are each a different fluorophore with a different light transmission wavelength than that of the other markers. The synthesized DNA molecule may include more than 3 unique defined sequences of nucleotides, each labeled with a marker having a distinct light transmission wavelength from that of the others. For example, the synthesized DNA molecule may include sequences of n, o, p, or q nucleotides. The synthesized DNA molecule may include as many unique sequences as may be labeled by unique markers having light transmission wavelengths that are each distinguishable from the other.

In an example, four different fluorophores may be used to label four different sequences of n nucleotides to increase the information density, as five different states may be coded within a sequence of n nucleotides (marker 1, marker 2, marker 3, marker 4, and no marker) as opposed to two different states (marker and no marker). This can be illustrated with the example of a molecule of DNA that has x information-carrying segments of n nucleotides. In the case of one marker, this piece of DNA can store 2× different states. In the case of 4 different markers, the same piece of DNA can store 5× different states. This increases the information density by a factor of e^{(x*ln(5/2))}.

Table 1 illustrates the benefit for different values for x in this example:

TABLE 1

| x | 1 | 2 | 3 | 4 | 5 | 6 |
|---|---|---|---|---|---|---|
| 2^{x} | 2 | 4 | 8 | 16 | 32 | 64 |
| 5^{x} | 5 | 25 | 125 | 625 | 3125 | 15625 |
| 5\hat{ }l/2\hat{ }x | 2.5 | 6 | 15 | 39 | 97 | 244 |

For the case of 6 segments and 4 different markers, the increase in information density is about 244. For the example of DNA with 1,000 base pairs, 8 nucleotides per segment and 12 spacer nucleotides between segments, x equals 50 and the advantage in information density when using 4 markers instead of 2 is e^{(50*ln(5/2)}, which is 7.9*10¹⁹.

In addition to optical markers, electrical markers including redox molecules may also be used to label the sequences of n nucleotides. Non-limiting examples of redox molecules that may be used as labels are metal-organic complexes, such as ferrocene and its derivatives, osmium and ruthenium complexes, conjugated organic molecules, such as tetrathiafulvalene, methylene blue, anthraquinone, phenothiazine, aminophenol, nitrophenol, erythrosine B, ATTO MB2, etc. The redox species undergo reversible oxidation-reduction reactions under applied electrical potential to enable the redox detection principle. To increase information density using several different markers, the redox molecules may be chosen in a way so that their oxidation and reduction potentials are different. When reading out the stored information, a reader device “reader” may apply the different reduction and oxidation potentials in time multiplex and measure at which combination of potentials an electric signal occurs. In the case of two different redox markers, three different states can be stored in a sequence of n nucleotides: marker 1, marker 2 and no marker. A DNA molecule that has two different types of markers incorporated may be synthesized by using one kind of nucleotide that is labeled by one kind of redox molecule and a different kind of nucleotide that is labeled by a different redox molecule. For example, all of the Adenines may be labeled with redox marker 1 and all of the Cytosines may be labeled with marker 2. Since in double stranded DNA Adenine hybridizes with Thymine and Cytosine hybridizes with Guanine, there would be no conflict. For the segment of n nucleotides that is marked by marker 1, one half-strand would consist of n Adenine molecules and the complementary half strand would be n un-marked Thymine molecules. For the segment of n nucleotides that is marked by marker 2, one half-strand would consist of n marked Cytosine molecules and the complimentary half strand would consist of n unmarked Guanine molecules. The storage of information would simply occur during DNA synthesis by only making Adenines conjugated to marker 1 available for DNA synthesis and only making Cytosines conjugated to marker 2 available for synthesis. It is also possible to store the information for a very long time in DNA with the target sequence of nucleotides without any redox molecules added. Before readout, this DNA can be copied and amplified using PCR. The nucleotides for making the copy of the original DNA may be conjugated to redox-molecules. This approach may decouple the shelf-life of the information storage in DNA from the shelf-life of redox-molecules. Incorporating the redox-molecules just before readout may be implemented in PCR-based readers for example.

In at least an embodiment, a method for storing and reading out information is provided. The method may include synthesizing a DNA molecule including at least one sequence of n nucleotides and at least one spacer sequence of s nucleotides. The method also includes encoding an element of information to be stored in a sequence of n nucleotides. The sequence of n nucleotides may be labeled with an optical marker that may be detected to facilitate readout of the stored information.

To read out the stored information, the synthesized DNA molecule with optically labeled sequences of n nucleotides may be pinned, as described in U.S. application 63/702,095, filed on Oct. 1, 2024, and incorporated by reference herein in its entirety. The labeled synthesized DNA molecule may be dissolved in a fluid. The fluid may be delivered to a surface of a microfluidic channel. A polymer may also be provided to the surface of the channel. The polymer may physically interact with the surface of the channel. For example, the polymer may coat the surface of the channel. The polymer may be capable of modifying an electroosmotic flow in the channel. The polymer may be a neutral and water-soluble polymer. The neutral and water-soluble polymer may be a polyvinylpyrrolidone polymer. The polyvinylpyrrolidone polymer may have a molecular weight greater than 100 kDa. The neutral and water-soluble polymer may alternatively be hydroxyethylcellulose, polyethylene glycol, or polyvinyl alcohol. The channel may have at least one dimension normal to the electric field of less than 5 microns. The electric field may be from 10 to 1,000 V/cm.

An electric field may be applied to the channel, including the contents of the channel which include the labeled synthesized DNA molecule, to promote a physical interaction between the labeled synthesized DNA molecule and the polymer. This physical interaction traps the labeled synthesized DNA molecule at the surface of the channel. The physical interaction between the labeled synthesized DNA molecule and the polymer may occur at a vertex of the labeled synthesized DNA molecule which may cause the ends of the labeled synthesized DNA molecule to stretch outwardly from the vertex upon application of the electric field. The ends of the labeled synthesized DNA molecule may stretch outwardly from the vertex in a direction opposite that of the electric field. The optically labeled sequences of n nucleotides in the labeled synthesized DNA molecule may be detected by a reader while the labeled synthesized DNA molecule is trapped and stretched in a field of view. The reader may for example be a camera capable of detecting the optical label. In this way, the reader may permit visualization of the labeled nucleotides in the sequence of n nucleotides. The information stored in the detected sequences of n nucleotides may then be read out by the reader.

Multiple synthesized DNA molecules may be included in a sample delivered to the channel. The synthesized DNA molecules may then be pinned within a controlled flow of fluid containing DNA and the information stored in the optically labelled sequences of n nucleotides may be optically read out while the DNA is held in place. After reading out the information from the pinned DNA, the DNA may be unpinned, and the fluid may be exchanged for a fresh volume of fluid containing a new batch of optically labeled synthesized DNA molecules to be read out next. The process of DNA pinning may be used to immobilize and linearize the labeled synthesized DNA molecules under a reader for high resolution readout. The fact that the labeled synthesized DNA molecules can be held still in place in a stretched conformation may give the reader enough time to collect enough information for a high readout quality at low cost.

As described in U.S. application 63/702,095, filed on Oct. 1, 2024, and incorporated by reference herein in its entirety, the term channel may include fluidic chambers in fluid communication with at least one port which may be used to fill the chamber with liquid. The channel may also be part of a microfluidic device that includes one or more channels each having a single input and a single output. The channel may be part of a device that includes multiple independent channels in which samples may be processed in parallel. The channel surface may be dielectric materials including oxides, a nitride such as silicon nitride, glass including borosilicate glass, or quartz. The channel may include a floor and one or more walls extending outward from the floor. The walls of the channel may be flat. The channel may have a depth from 500 nm to 3 μm which may enhance the acquisition of high quality images of trapped labeled synthesized DNA molecules by the reader. In other embodiments, the channel may have a depth greater than 3 μm.

The transport of reagents and/or liquid in a system or device including the channel may be accomplished via electromigration, electroosmotic flow, pressure-driven flow, dielectrophoresis, magnetophoresis, electrowetting, induced charge electroosmotic flows, alternating current (AC) electrokinetic flows, gravity-driven flows, and surface-tension driven flows for example.

One type of marker (resulting in a digital encoding) or markers with different “colors”, meaning emission wavelengths, (resulting in a multivariant encoding) may be used. The reader for optical markers may be a camera with a suitable detection range around the emission wavelengths of the chosen markers. As described above, the labeled synthesized DNA molecules may be held in place long enough to gather sufficient information, in that case light, to expose a high quality picture of the DNA. In this way, stored information may be quickly and accurately read out without having to use costly highly specialized sequencers.

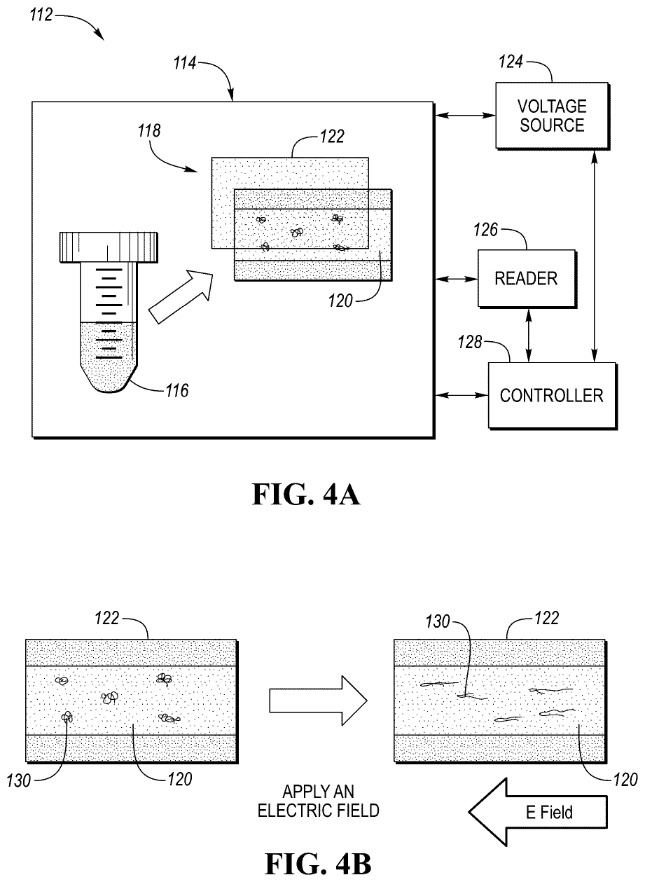

FIG. 4A illustrates a system 112 for storing and reading out information according to an embodiment. The system 112 may include a device 114 having at least one reservoir 116 and a fluidic chip 118 having at least one channel 120. The at least one channel 120 may be in fluid communication with the at least one reservoir 116 so that a sample is capable of flowing through the at least one channel 120. The device 114 may also have a channel cover 122 which may be transparent to the emission wavelengths of the chosen markers. The system may also include a voltage source 124 capable of applying an electric field to the at least one channel 120 and at least one reader 126 capable of detecting signals from the device 114. The at least one reader 126 may be a camera with a suitable detection range around the emission wavelengths of the chosen markers. The system 112 may also include a controller 128 programmed to direct the flow of a polymer to the device 114 to promote delivery of the polymer to the surface of the at least one channel 120 so that the polymer is capable of physically interacting with the surface of the at least one channel 120. The controller 128 may also be programmed to direct the flow of a sample including a labeled synthesized DNA molecule 130 having at least one sequence of n nucleotides in which information is encoded and at least one spacer sequence of s nucleotides from the at least one reservoir 116 to the at least one channel 120. The controller 128 may also be programmed to generate a voltage difference between an electrode and at least one other electrode to create an electric field in the channel 120 of the device 114 and promote a physical interaction between the labeled synthesized DNA molecule 130 and the polymer to trap the labeled synthesized DNA molecule 130 at the surface of the channel 120. The controller 128 may be programmed to interact with the at least one reader 126 to collect, record, and store a signal received from the at least one reader 126, and to process the signal received from the at least one reader 126. In this way, the information encoded in the sequences of n nucleotides may be read out. For example, the reader 126 may be a camera that captures an image of a number of labeled synthesized DNA molecules 130 trapped in a field of view. The markers on the labeled synthesized DNA molecules may then be decoded and the information may be read out without having to use costly sequencers.

The controller 128 may be further programmed to lower the magnitude of the electric field applied to the channel to promote release of the labeled synthesized DNA molecule 130 from the physical interaction with the polymer so that the labeled synthesized DNA molecule 130 is cleared from the channel 120. The controller 128 may be programmed to then raise the magnitude of the electric field applied to the channel 120 so that a subsequent batch of labeled synthesized DNA molecules 130 may be trapped and the information encoded in the sequences of n nucleotides may be read out.

FIG. 4B illustrates labeled synthesized DNA molecules 130 pinned by a physical interaction with a polymer upon application of an electric field. After a sample including a labeled synthesized DNA molecule 130 having at least one sequence of n nucleotides in which information is encoded and at least one spacer sequence of s nucleotides is directed from the at least one reservoir 116 to the at least one channel 120, a voltage difference between an electrode and at least one other electrode is generated by the voltage source 124 to create an electric field in the channel 120 of the device 114. The electric field promotes a physical interaction between the labeled synthesized DNA molecule 130 and the polymer which traps the labeled synthesized DNA molecule 130 at the surface of the channel 120. The labeled synthesized DNA molecule 130 is pinned at a vertex of the labeled synthesized DNA molecule 130. Each end of the labeled synthesized DNA molecule 130 stretches away from the vertex in a direction opposite that of the electric field. The label (also referred to as a marker) on the sequence of n nucleotides on the labeled synthesized DNA molecule 130 may then be detected by the reader 126 so that the information encoded in the sequence of n nucleotides may be read out.

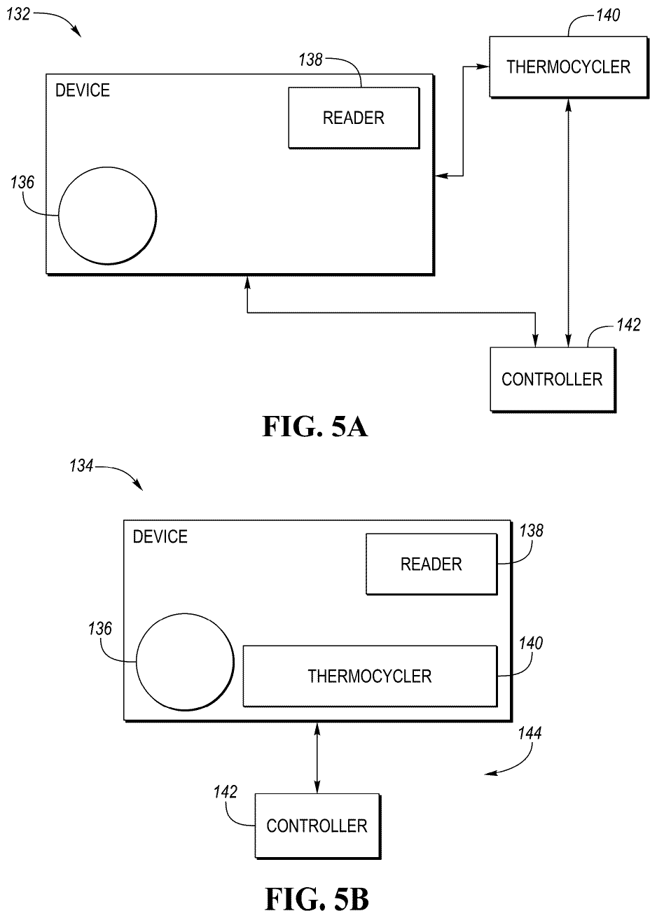

FIG. 5A illustrates a system 132 for storing and reading out information according to an embodiment. The system may include a device 134 having at least one reservoir 136 sized to receive a sample including a synthesized DNA molecule having at least one sequence of n nucleotides in which information is encoded and at least one sequence of s nucleotides. The device 134 may also include a reader 138. The reader 138 may include a microfluidic chip. The reader 138 may have at least one electrochemical nanosensor. The electrochemical nanoelectrode sensor may have two electrodes separated by a dielectric layer defining a sensing zone between the electrodes. The reader 138 may also include readout electronics (not shown). The readout electronics may be similar to those described in US 20190137435A1. The system may also include a thermocycler 140. The thermocycler 140 may be a system component that is separate and distinct from the device 134 that is capable of physically and electronically interacting with the device. The system may additionally include a controller 142 programmed to deliver the sample to the thermocycler 140. Alternatively, the sample may be delivered to the thermocycler 140 by hand. The controller 142 may also be programmed to direct the thermocycler 140 to execute a PCR-based reaction. For example, the controller 142 may be programmed to direct the thermocycler 140 to execute a PCR-based reaction that promotes labeling of the sequences of n nucleotides in the synthesized DNA molecule with electroactive labels. The controller 142 may then be programmed to deliver the labeled synthesized DNA molecule to the reader 140 where the controller 142 is programmed to direct current through the electrodes to induce electron flow between the electrodes to produce a measurable electrical signal when an electroactive label is present in the sensing zone. In this way, the electroactive labels on the nucleotides of the sequence of n nucleotides may be detected. The element of information encoded in the sequence of n nucleotides may therefore be read out.

FIG. 5B illustrates a system 144 for storing and reading out information according to an embodiment. The system may include a device 134 having at least one reservoir 136 sized to receive a sample including a synthesized DNA molecule having at least one sequence of n nucleotides in which information is encoded and at least one sequence of s nucleotides. The device 134 may also include a reader 138. The reader 138 may include a microfluidic chip. The reader 138 may have at least one electrochemical nanosensor. The electrochemical nanoelectrode sensor may have two electrodes separated by a dielectric layer defining a sensing zone between the electrodes. The reader 138 may also include readout electronics (not shown). The readout electronics may be similar to those described in US 20190137435A1. The device may also include a thermocycler 140. The system may additionally include a controller 142 programmed to deliver the sample to the thermocycler 140. Alternatively, the sample may be delivered to the thermocycler 140 by hand. The controller 142 may also be programmed to direct the thermocycler 140 to execute a PCR-based reaction. For example, the controller 142 may be programmed to direct the thermocycler 140 to execute a PCR-based reaction that promotes labeling of the sequences of n nucleotides in the synthesized DNA molecule with electroactive labels. The controller 142 may then be programmed to deliver the labeled synthesized DNA molecule to the reader 140 where the controller 142 is programmed to direct current through the electrodes to induce electron flow between the electrodes to produce a measurable electrical signal when an electroactive label is present in the sensing zone. In this way, the electroactive labels on the nucleotides of the sequence of n nucleotides may be detected. The element of information encoded in the sequence of n nucleotides may therefore be read out.

### Examples: Incorporating Redox-Labeled Nucleotides into a Sequence of n Nucleotides

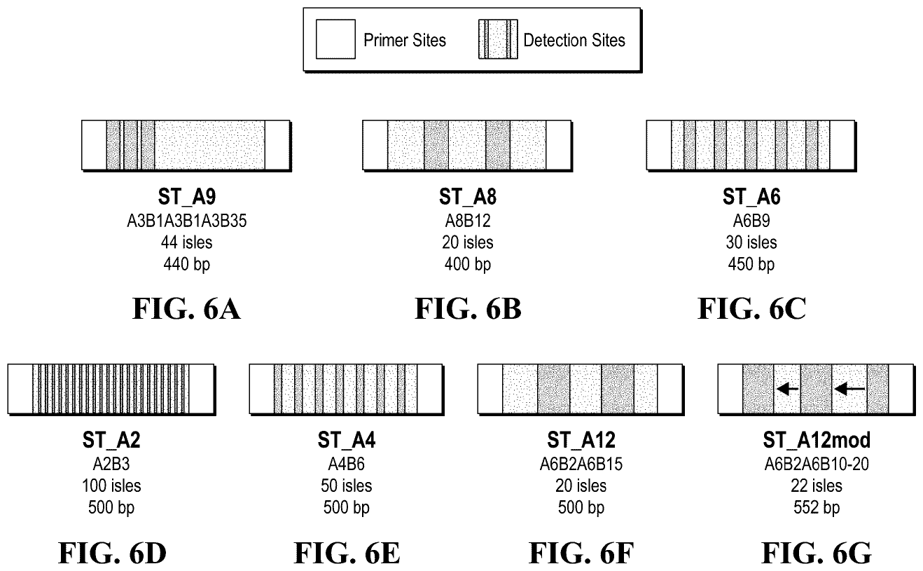

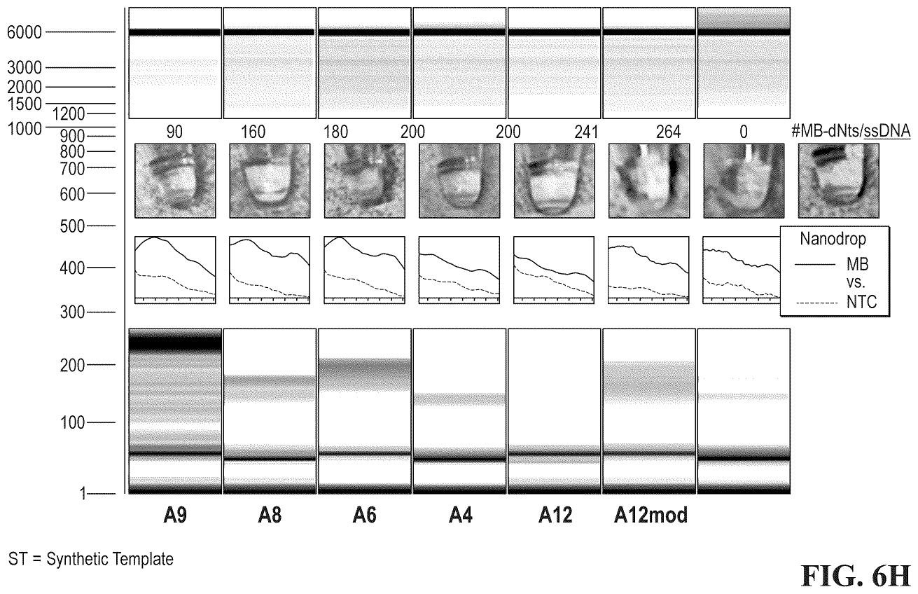

Template DNA molecules of varying length, having varying numbers of sequences of n nucleotides (“detection sites”) were synthesized (FIGS. 6A through 6G). Each template had a primer recognition sequence on each end to facilitate PCR-based amplification. Each template had a number of sequences of n nucleotides, each having a different number of nucleotides having an adenine base (“A #”). Methylene blue labeled dUTP nucleotides were successfully incorporated into the sequences of n nucleotides in each template DNA molecule (FIG. 6H). NTC signifies a no-template control that should give no signal. The templates in FIGS. 6F and 6G each included 2 stretches of 6 labeled nucleotides separated by two unlabeled nucleotides.

While exemplary embodiments are described above, it is not intended that these embodiments describe all possible forms encompassed by the claims. The words used in the specification are words of description rather than limitation, and it is understood that various changes can be made without departing from the spirit and scope of the disclosure. As previously described, the features of various embodiments can be combined to form further embodiments of the invention that may not be explicitly described or illustrated. While various embodiments could have been described as providing advantages or being preferred over other embodiments or prior art implementations with respect to one or more desired characteristics, those of ordinary skill in the art recognize that one or more features or characteristics can be compromised to achieve desired overall system attributes, which depend on the specific application and implementation. These attributes can include, but are not limited to cost, strength, durability, life cycle cost, marketability, appearance, packaging, size, serviceability, weight, manufacturability, ease of assembly, etc. As such, to the extent any embodiments are described as less desirable than other embodiments or prior art implementations with respect to one or more characteristics, these embodiments are not outside the scope of the disclosure and can be desirable for particular applications.

What is claimed is:

1. A method for storing information in a DNA molecule comprising:
providing a DNA molecule having at least one sequence of n nucleotides and at least one sequence of s nucleotides; and
encoding an element of information in the sequence of n nucleotides to store the element of information in the DNA molecule.

2. The method of claim 1, wherein the sequence of n nucleotides consists of a defined sequence of nucleotides.

3. The method of claim 1, further comprising labeling the sequence of n nucleotides with at least one marker to obtain a labeled DNA molecule.

4. A method for reading out information from a DNA molecule comprising:
providing a DNA molecule having at least one sequence of n nucleotides and at least one sequence of s nucleotides, wherein the at least one sequence of n nucleotides is labeled with at least one marker to obtain a labeled DNA molecule;
detecting the at least one marker; and
reading out an element of information encoded in the labeled sequence of n nucleotides.

5. The method of claim 4, wherein the sequence of n nucleotides includes nucleotides each having the same nucleotide base.

6. The method of claim 4 wherein the marker is detected by:
providing the labeled synthesized DNA molecule in a fluid sample to a surface of a microfluidic channel;
delivering a polymer to the surface of the microfluidic channel;
physically interacting the polymer with the surface of the microfluidic channel;
applying an electric field to the channel to promote a physical interaction between the synthesized DNA molecule and the polymer so that the synthesized DNA molecule is trapped at the surface of the channel; and
visualizing the trapped labeled synthesized DNA molecule.

7. The method of claim 6, wherein the marker is an optical marker.

8. The method of claim 6, wherein the polymer is a neutral and water-soluble polymer.

9. The method of claim 8, wherein the polymer is a polyvinylpyrrolidone polymer having a molecular weight greater than 100 kDa.

10. The method of claim 9, wherein the electric field is from 10 to 1,000 V/cm.

11. The method of claim 4, wherein the labeled synthesized DNA molecule includes more than one sequence of n nucleotides wherein at least one of the more than one sequence of n nucleotides is labeled with a distinct marker having a distinct emission wavelength from that of the other of the more than one sequence of n nucleotides.

12. The method of claim 11, wherein at least one of the more than one sequence of n nucleotides has a different length than at least one other of the more than one sequence of n nucleotides.

13. The method of claim 5, wherein the nucleotides in the sequence of nucleotides are labeled with an electrical marker.

14. The method of claim 13, wherein the marker is detected by:
applying an electrical potential to the labeled synthesized DNA molecule to promote a reversible oxidation-reduction reaction of the marker; and
measuring the oxidation and/or reduction potentials to identify the marker.

15. The method of claim 13, wherein the labeled synthesized DNA molecule includes more than one sequence of n nucleotides each labeled with a distinct marker having a distinct electrical signal.

16. The method of claim 15, wherein detecting the marker includes applying the different reduction and oxidation potentials generated by the distinct markers in time multiplex and measuring a combination of potentials at which an electric signal occurs.

17. A system for reading out information from a DNA molecule comprising:
a device having at least one reservoir; a fluidic chip with at least one channel, the at least one channel in fluid communication with the at least one reservoir; and a channel cover;
a voltage source capable of applying an electric field to the at least one channel;
at least one reader capable of detecting signals from the device; and
a controller programmed to:
direct the flow of a polymer to the device to promote delivery of the polymer to the surface of the at least one channel;
direct the flow of a sample including a labeled synthesized DNA molecule having at least one sequence of n nucleotides in which information is encoded and at least one spacer sequence of s nucleotides from the at least one reservoir to the at least one channel;
generate a voltage difference between an electrode and at least one other electrode to create an electric field in the channel of the device and promote a physical interaction between the labeled synthesized DNA molecule and the polymer to trap the labeled synthesized DNA molecule at the surface of the channel; and
interact with the at least one reader to collect, record, store and process a signal received from the at least one reader.

18. The system of claim 17, wherein the sequence of n nucleotides is labeled with an optical marker.

19. The system of claim 18, wherein the polymer is a neutral and water-soluble polymer.

20. The system of claim 18, wherein the electric field is from 10 to 1,000 V/cm.

21. The system of claim 20, wherein the labeled synthesized DNA molecule includes more than one sequence of n nucleotides, wherein at least one sequence of n nucleotides is labeled with a distinct marker having a distinct emission wavelength from at least one other of the more than one sequence of n nucleotides.
